(19) United States
(12) Patent Application Publication
(10) Pub. No.: US 2010/0121146 A1
(43) Pub. Date: May 13, 2010

(54) SCANNING ENDOSCOPE, SCANNING ENDOSCOPE PROCESSOR, AND SCANNING ENDOSCOPE APPARATUS

(75) Inventor: Hideo SUGIMOTO, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/614,685

(22) Filed: Nov. 9, 2009

(30) Foreign Application Priority Data
Nov. 11, 2008 (JP) 2008-289209

(51) Int. Cl. A61B 1/06

(52) U.S. Cl. 600/117; 600/173

(57) ABSTRACT

A scanning endoscope comprising a first transmitter, an actuator, a first mirror, and a second mirror, is provided. The first transmitter emits a beam of radiant light from an emission end. The actuator moves the emission end along a spiral course. The first mirror is arranged from the emission end toward a first direction. The first mirror comprises a first reflection surface around the first direction. The first reflection surface reflects the radiant light emitted from the first transmitter. The second mirror is arranged around the first reflection surface. The second mirror comprises a second reflection surface. The second reflection surface reflects the radiant light reflected by the first mirror in a direction that includes the first direction as a positive vector toward any points on the first line.

### BACKGROUND OF THE INVENTION

1. Field of the Invention

The present invention relates to reducing distortion appearing around the center of a spiral scanning course when using a scanning endoscope that scans a subject with illumination light along the spiral scanning course.

2. Description of the Related Art

A scanning endoscope, which photographs and/or films an optical image of an observation area by scanning the observation area with light shined on a minute point in the area and successively capturing reflected light at the illuminated points, is known. In a general scanning endoscope, light for illumination is transmitted through an optical fiber from a stationary incident end to a movable emission end and a scanning operation is carried out by successively moving the emission end of the optical fiber.

For the purpose of quick and stable scanning, Japanese Patent No. 3943927 discloses that the emission end of the optical fiber is moved along a spiral course. It is possible to reproduce an image with little distortion by spirally moving the emission end so that the distance from the center of the spiral course to the position of the emission end of the optical fiber increases in proportion to the amount of elapsed time since the emission end began moving away from the center on its spiral course.

It is possible to move the emission end along the spiral course in a stable manner when the emission end is a sufficient distance away from the center of the spiral course. However, it is difficult to circulate the emission end and increase the radius of the circulation when the emission end is near the center of the spiral course. Accordingly, distortion appears near the point corresponding to the center of the spiral course.

### SUMMARY OF THE INVENTION

Therefore, an object of the present invention is to provide a scanning endoscope that scans an observation area with light along a spiral scanning course while reducing the amount of distortion that appears in a reproduced image near the point corresponding to the center of the spiral course.

According to the present invention, a scanning endoscope, comprising a first transmitter, an actuator, a first mirror, and a second mirror, is provided. The first transmitter emits a beam of radiant light from an emission end. The beam of radiant light is shined on an observation area. The actuator moves the emission end along a spiral course from a predetermined standard point. The first mirror is arranged from the emission end toward a first direction when the emission end is on the standard point. The radiant light is emitted in the first direction from the emission end when the emission end is on the standard point. The first mirror comprises a first reflection surface around the first direction. The distance between a first position on a first line to any second position on the first reflection surface increases as the first position is moved in the first direction. The standard point is on the first line. The first line is parallel to the first direction. The first reflection surface reflects the radiant light emitted from the first transmitter. A line connecting the first and second positions is perpendicular to the first line. The second mirror is arranged around the first reflection surface. The second mirror comprises a second reflection surface. The second reflection surface reflects the radiant light reflected by the first mirror in a direction that includes the first direction as a positive vector toward any points on the first line.

According to the present invention, a scanning endoscope processor, comprising a light source, a light receiver, an image processor, and a first controller, is provided. The light source supplies the radiant light to the first transmitter of the scanning endoscope. The light receiver detects and receives various amounts of reflected light at the observation area illuminated with the radiant light. The image processor produces an image corresponding to the observation area on the basis of the amounts of the reflected light detected by the light receiver. The first controller suspends the production of an image at the image processor when the emission end is within a first area of which its center is the standard point and of which its radius is a first length. The first controller orders the image processor to produce the image when the emission end is outside of the first area.

According to the present invention, a scanning endoscope apparatus, comprising the scanning endoscope and the scanning endoscope processor, is provided.

### BRIEF DESCRIPTION OF THE DRAWINGS

The objects and advantages of the present invention will be better understood from the following description, with reference to the accompanying drawings in which:

FIG. 1 illustrates the schematic appearance of a scanning endoscope apparatus comprising a scanning endoscope and a scanning endoscope processor of the first and second embodiments of the present invention;

FIG. 2 is a block diagram schematically showing the internal structure of the scanning endoscope processor of the first embodiment;

FIG. 3 is a block diagram schematically showing the internal structure of the light-source unit of the first embodiment;

FIG. 4 is a block diagram schematically showing the internal structure of the scanning endoscope of the first embodiment;

FIG. 5 is a sectional view of the hollow tube and the optical unit along the axis direction of the illumination fiber for illustration of the arrangements of the optical unit and the illumination fiber in the first embodiment;

FIG. 6 is a sectional view of the fiber actuator along the axis direction of the illumination fiber for illustration of the structure of the fiber actuator in the first and second embodiments;

FIG. 7 is a front view of the fiber actuator in the first and second embodiments as seen from the emission end of the illumination fiber;

FIG. 8 is a perspective view of the fiber actuator in the first and second embodiments;

FIG. 9 is a graph illustrating the changing position of the emission end moving from the standard point along the first and second bending directions in the first and second embodiments;

FIG. 10 illustrates a spiral course along which the emission end of the illumination fiber is moved by the fiber actuator;

FIG. 11 is a perspective view of the second mirror in the first embodiment;

FIG. 12 is a perspective view of the first mirror in the first and second embodiments;

FIG. 13 illustrates the point on the first mirror illuminated with the white laser beam when the emission end of the illumination fiber is moved along the first circumference;

FIG. 14 illustrates the locus of the white laser beam emitted from the illumination fiber for explaining the conditions regarding the shapes of the first and second mirrors;

FIG. 15 illustrates the white laser beam emitted from the condenser lens;

FIG. 16 is a block diagram schematically showing the internal structure of the scanning endoscope processor of the second embodiment;

FIG. 17 is a block diagram schematically showing the internal structure of the light-source unit of the second embodiment;

FIG. 18 is a block diagram schematically showing the internal structure of the scanning endoscope of the second embodiment;

FIG. 19 is a perspective view of the second mirror unit in the second embodiment;

FIG. 20 is a perspective view of the second mirror in the second embodiment; and

FIG. 21 is a block diagram schematically showing the internal structure of the position estimation unit of the second embodiment.

### DESCRIPTION OF THE PREFERRED EMBODIMENTS

The present invention is described below with reference to the embodiment shown in the drawings.

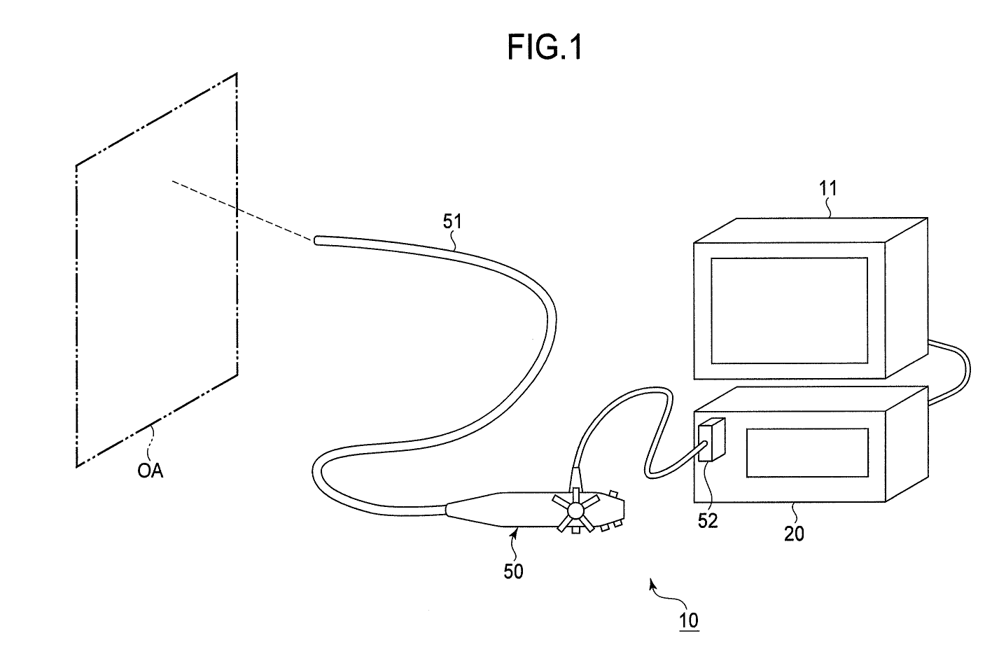

In FIG. 1, the scanning endoscope apparatus 10 comprises a scanning endoscope processor 20, a scanning endoscope 50, and a monitor 11. The scanning endoscope processor 20 is connected to the scanning endoscope 50 and the monitor 11.

Hereinafter, an emission end of an illumination fiber (not depicted in FIG. 1) and incident ends of image fibers (not depicted in FIG. 1) are ends mounted in the distal end of the insertion tube 51 of the scanning endoscope 50. In addition, an incident end of the illumination fiber and emission ends of the image fibers are ends mounted in a connector 52 that connects to the scanning endoscope processor 20.

The scanning endoscope processor 20 provides light that is shined on an observation area (see “OA” in FIG. 1). The light emitted from the scanning endoscope processor 20 is transmitted to the distal end of the insertion tube 51 through the illumination fiber (first transmitter), and is directed towards one point in the observation area. Light reflected from the illuminated point is transmitted from the distal end of the insertion tube 51 to the scanning endoscope processor 20.

The direction of the emission end of the illumination fiber is changed by a fiber actuator (not depicted in FIG. 1). By changing the direction, the observation area is scanned with the light emitted from the illumination fiber. The fiber actuator is controlled by the scanning endoscope processor 20.

The scanning endoscope processor 20 receives reflected light that is scattered at the illuminated point, and generates a pixel signal according to the amount of received light. One frame of an image signal is generated by generating pixel signals corresponding to the illuminated points dispersed throughout the observation area. The generated image signal is transmitted to the monitor 11, where an image corresponding to the received image signal is displayed.

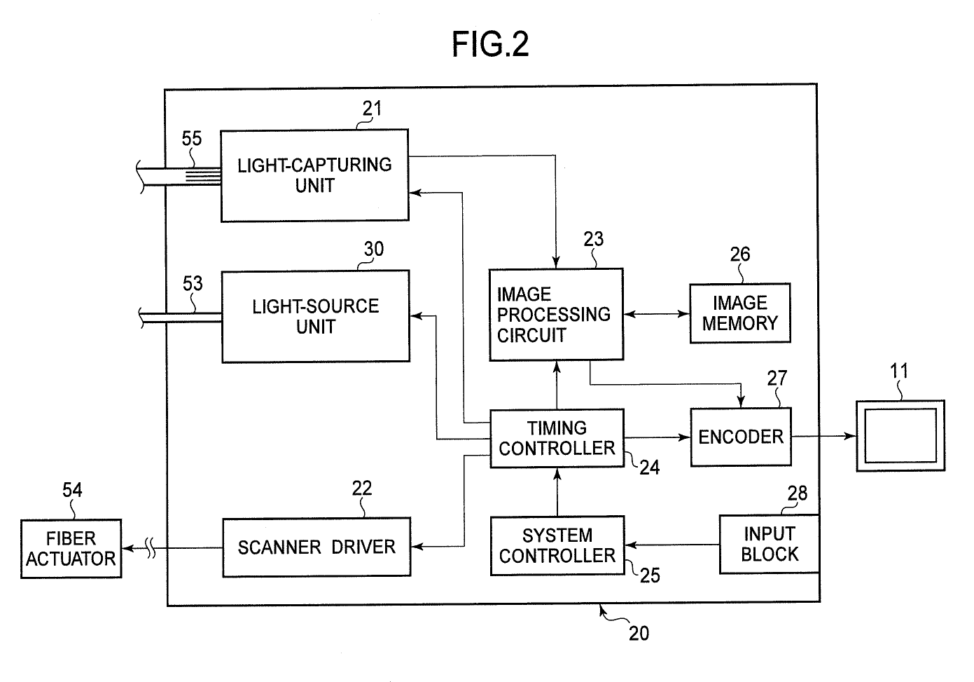

As shown in FIG. 2, the scanning endoscope processor 20 comprises a light-source unit 30, a light-capturing unit 21, a scanner driver 22, an image processing circuit 23, a timing controller 24, a system controller 25, and other components.

As described later, the light-source unit 30 provides the illumination fiber 53 with white light (radiant light) to illuminate an observation area. The scanning driver 22 controls the fiber actuator 54 to move the emission end of the illumination fiber 53. The reflected light at the illuminated point is transmitted to the scanning endoscope processor 20 by the scanning endoscope 50. The transmitted light is made incident on the light-capturing unit 21.

The light-capturing unit 21 generates a pixel signal according to the amount of the reflected light. The pixel signal is transmitted to the image processing circuit 23, which stores the received pixel signal in the image memory 26. Once pixel signals corresponding to the illuminated points dispersed throughout the observation area have been stored, the image processing circuit 23 carries out predetermined image processing on the pixel signals, and then one frame of the image signal is transmitted to the monitor 11 via the encoder 27.

By connecting the scanning endoscope 50 to the scanning endoscope processor 20, optical connections are made between the light-source unit 30 and the illumination fiber 53 mounted in the scanning endoscope 50, and between the light-capturing unit 21 and the image fibers 55. In addition, by connecting the scanning endoscope 50 to the scanning endoscope processor 20, the fiber actuator 54 mounted in the scanning endoscope 50 is electrically connected to the scanning driver 22.

The timing for carrying out the operations of the light-source unit 30, the light-capturing unit 21, the image processing circuit 23, the scanning driver 22, and the encoder 27 is controlled by the timing controller 24. In addition, the timing controller 24 and other components of the endoscope apparatus 10 are controlled by the system controller 25. A user can input some commands to the input block 28, which comprises a front panel (not depicted) and other mechanisms.

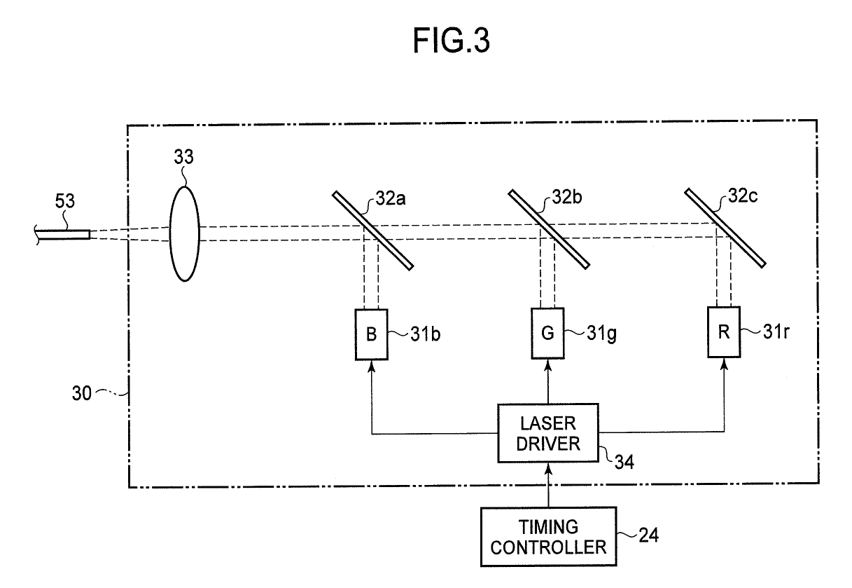

As shown in FIG. 3, the light-source unit 30 comprises a red laser 31r, a green laser 31g, a blue laser 31b, first to third filters 32a, 32b, and 32c, a condenser lens 33, a laser driver 34, and other components.

The red, green, and blue lasers 31r, 31g, and 31b emit red, green, and blue laser beams, respectively.

The first filter 32a reflects the band of blue light that the blue laser 31b emits, and transmits the other bands. The second filter 32b reflects the band of green light that the green laser 31g emits, and transmits the other bands. The third filter 32c reflects the band of red light that the red laser 31r emits, and transmits the other bands.

The condenser lens 33, the first filter 32a, the second filter 32b, the third filter 32c are arranged in the incident direction of the incident end of the illumination fiber 53, which is connected to the light-source unit 30. The first to third filters 32a, 32b and 32c are fixed so that the surfaces of the filters are inclined by 45 degrees against the axis direction of the illumination fiber 53.

The blue laser 31b is mounted so that the blue laser beam emitted by the blue laser 31b is reflected by the first filter 32a and made incident on the incident end of the illumination fiber 53.

The green laser 31g is mounted so that the green laser beam emitted by the green laser 31g is reflected by the second filter 32b, transmitted by the first filter 32a, and made incident on the incident end of the illumination fiber 53.

The red laser 31r is mounted so that the red laser beam emitted by the red laser 31r is reflected by the third filter 32c, transmitted by the first and second filters 32a and 32b, and made incident on the incident end of the illumination fiber 53.

The blue, green, and red laser beams are condensed by the condenser lens 33, and made incident on the incident end of the illumination fiber 53.

Upon observing a real-time image in the peripheral area of the insertion tube 51, the red, green, and blue laser beams are mixed into a white laser beam, which is supplied to the illumination fiber 53.

The laser driver 34 drives the red, green, and blue lasers 31r, 31g, and 31b. In addition, on the basis of the control of the timing controller 24, the laser driver 34 controls the light-on and -off timing for the lasers 31r, 31g, and 31b.

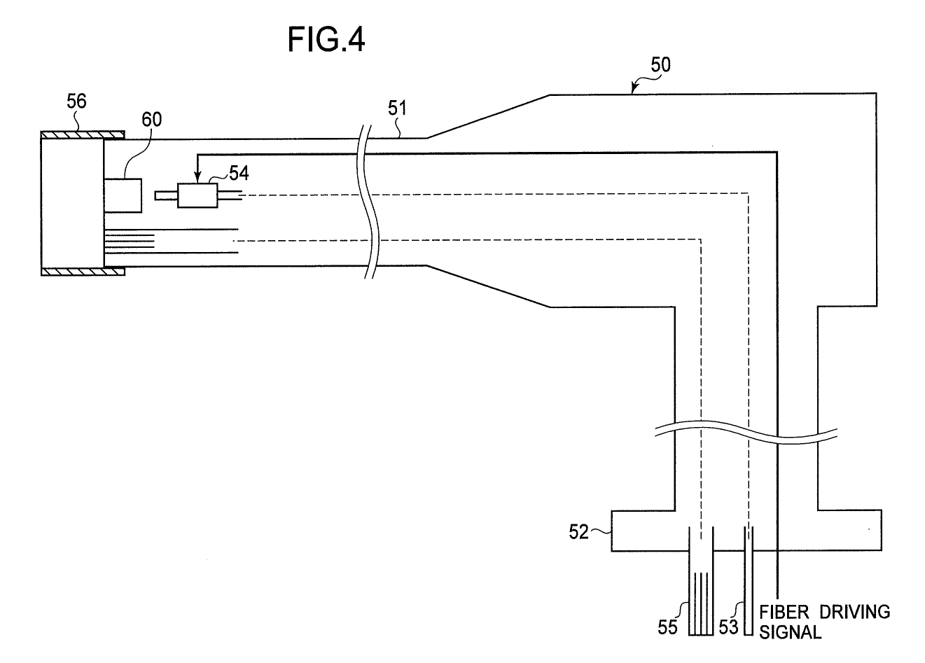

Next, the structure of the scanning endoscope 50 is explained. As shown in FIG. 4, the scanning endoscope 50 comprises the illumination fiber 53, the fiber actuator 54, the image fibers 55, an optical unit 60, a hood 56 (guide), and other components.

The illumination fiber 53 and the image fibers 55 are arranged inside the scanning endoscope 50 from the connector 52 to the distal end of the insertion tube 51. As described above, the white laser beam emitted by the light-source unit 30 is incident on the incident end of the illumination fiber 53. The incident white laser beam is transmitted to the emission end of the illumination fiber 53.

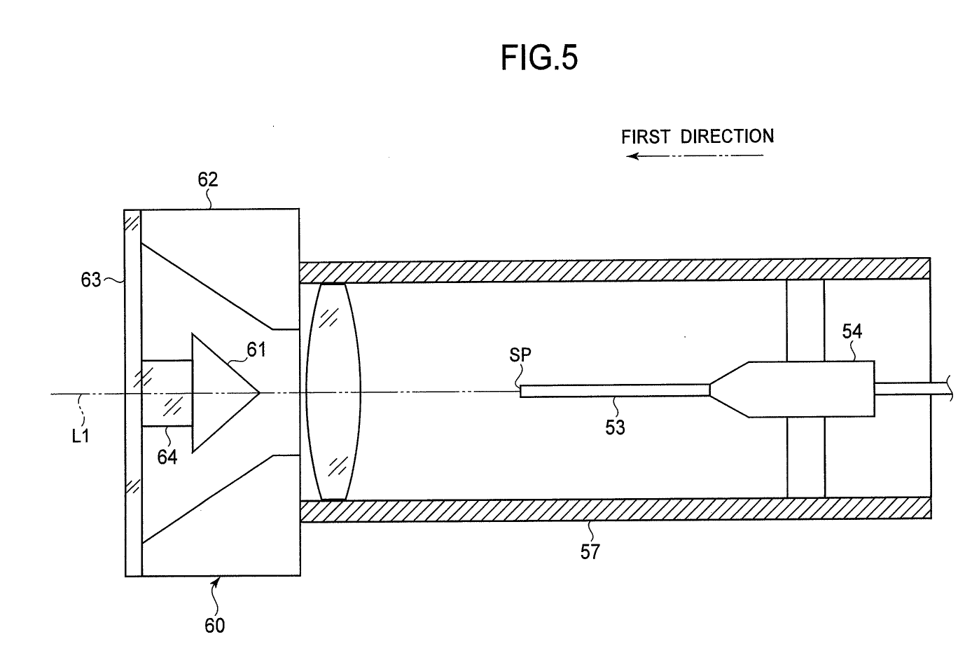

A solid hollow tube 57 is mounted at the distal end of the insertion tube 51 (see FIG. 5). The hollow tube 57 is positioned so that the axis directions of the distal end of the insertion tube 51 and the hollow tube 57 are parallel.

As shown in FIG. 5, the illumination fiber 53 is supported inside the hollow tube 57 by the fiber actuator 54. The illumination fiber 53 is positioned in the hollow tube 57 so that the axis direction of the hollow tube 57 is parallel to a first direction, which is an axis direction, of the insertion tube 51 that is not moved by the fiber actuator 54.

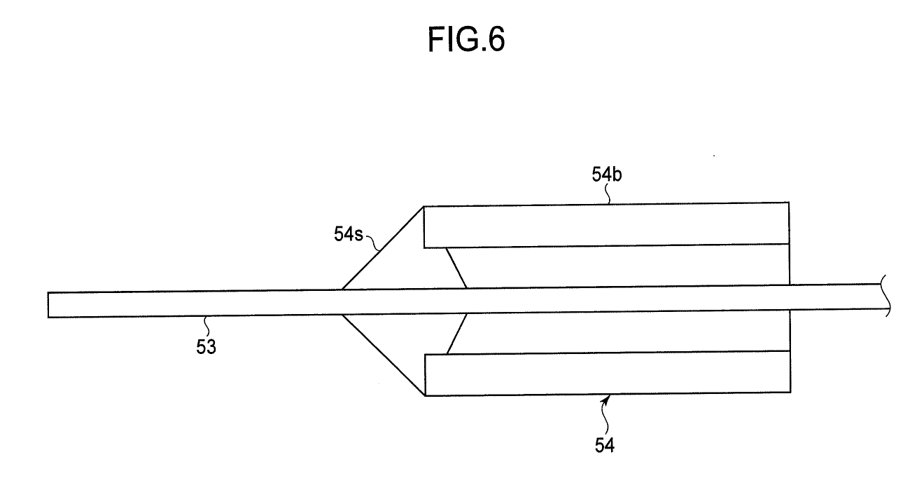

As shown in FIG. 6, the fiber actuator 54 comprises a supporting block 54s and a bending block 54b. The bending block 54b is shaped cylindrically. The illumination fiber 53 is inserted through the cylindrical bending block 54b. The illumination fiber 53 is supported at the forward end of the bending block 54b nearest the distal end of the insertion tube 51 by the supporting block 54s.

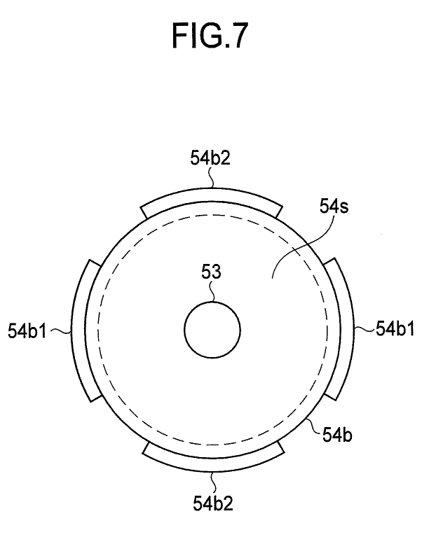

As shown in FIG. 7, first and second bending elements 54b1 and 54b2 are fixed on the bending block 54b. The first and second bending elements 54b1 and 54b2 are pairs of two piezoelectric elements. In addition, the first and second bending elements 54b1 and 54b2 expand and contract along the axis direction of the cylindrical bending block 54b on the basis of a fiber driving signal transmitted from the scanner driver 22.

Two piezoelectric elements that constitute the first bending element 54b1 are fixed on the outside surface of the cylindrical bending block 54b so that the axis of the cylindrical bending block 54b is between the piezoelectric elements. In addition, two piezoelectric elements that constitute the second bending element 54b2 are fixed on the outside surface of the cylindrical bending block 54b at a location that is 90 degrees circumferentially from the first bending element 54b1 around the axis of the cylindrical bending block 54b.

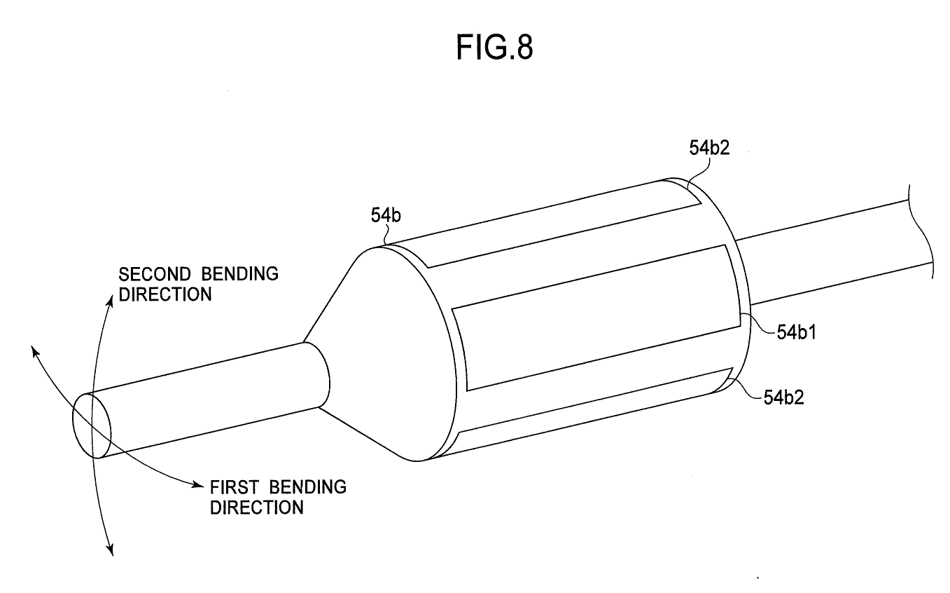

As shown in FIG. 8, the bending block 54b bends along a first bending direction by expanding one of the piezoelectric elements that constitute the first bending element 54b1 and contracting the other at the same time. The piezoelectric elements constituting the first bending element 54b1 are arranged along the first bending direction.

In addition, the bending block 54b bends along a second bending direction by expanding one of the piezoelectric elements that constitute the second bending element 54b2 and contracting the other at the same time. The piezoelectric elements constituting the second bending element 54b2 are arranged along the second bending direction.

The side of illumination fiber 53 is pushed along the first and/or second bending directions by the bending block 54b via the supporting block 54s, and the illumination fiber 53 bends toward the first and/or second bending directions, which are perpendicular to the axis direction of the illumination fiber 53. The emission end of the illumination fiber 53 is moved by bending the illumination fiber 53.

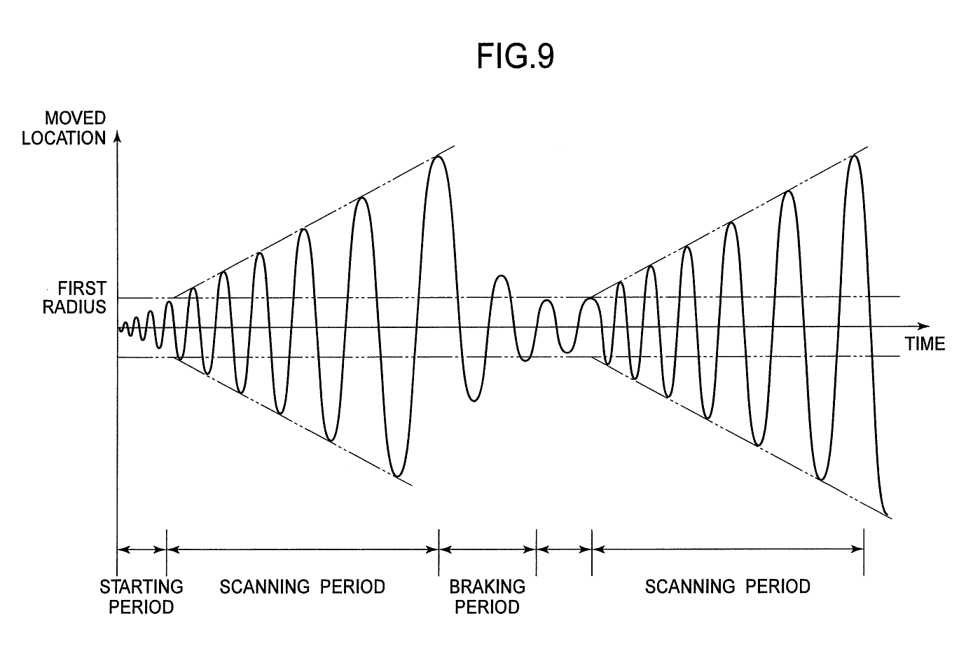

As shown in FIG. 9, the emission end of the illumination fiber 53 is moved so that the emission end vibrates along the first and second bending directions at amplitudes that are repetitively increased and decreased. The frequencies of the vibration along the first and second bending directions are adjusted to be equal. In addition, the period to increase and to decrease the amplitudes of the vibration along the first and second bending directions are synchronized. Further, phases of the vibration along the first and second bending directions are shifted by 90 degrees.

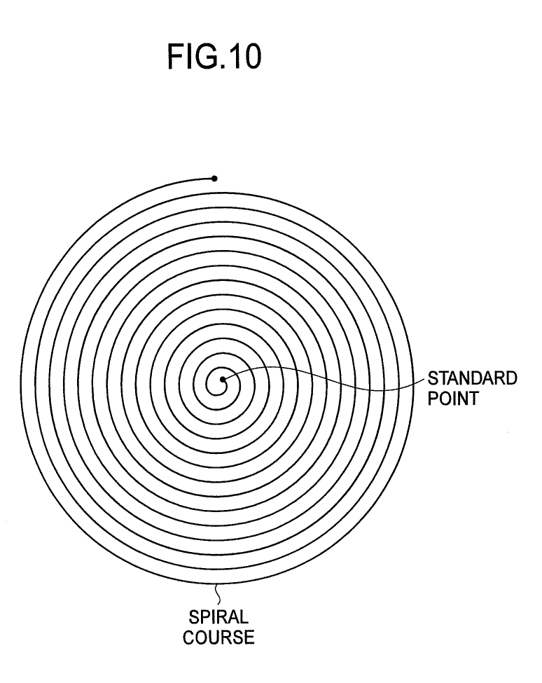

By vibrating the emission end of the illumination fiber 53 along the first and second bending directions as described above, the emission end traces the spiral course shown in FIG. 10, and the observation area is scanned with the white laser beam.

The position of the emission end of the illumination fiber 53 when the illumination fiber 53 is not bent is defined as a standard point. As described later, while the emission end is vibrated with increasing amplitude starting from the circulation of the emission end on a predetermined circumference (see “scanning period” in FIG. 9), illumination of the observation area with the white laser beam and generation of pixel signals are carried out.

In addition, when the amplitude reaches a maximum among the predetermined range, one scanning operation for producing one image terminates. After termination of a scanning operation, the emission end of the illumination fiber 53 is returned along the predetermined circumference by vibrating the emission end along the first and second bending directions at decreasing amplitudes during a braking period, as shown in FIG. 9. When the emission end is moved along the predetermined circumference, it is the beginning of a scanning operation for generating another image.

The optical unit 60 is mounted on the end of the hollow tube 57 in the axis direction, which is in the emission direction of light from the emission end that is positioned on the standard point. The optical unit 60 comprises first and second mirrors 61 and 62, and a mirror fixing plate 63 (see FIG. 5).

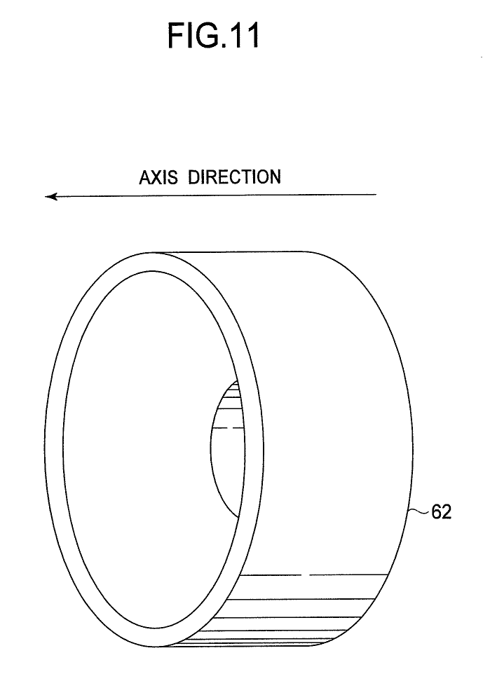

As shown in FIG. 11, the second mirror 62 has a hollow tube shape so that the inside surface of the hollow tube is a conical surface and the internal diameter of the hollow tube increases along the axis direction of the hollow tube shape. On the inside surface of the second mirror 62 is formed a second reflection surface that reflects the white laser beam emitted from the light-source unit 30.

The mirror fixing plate 63 is attached to the second mirror 62 on the end having the greater internal diameter (see FIG. 5). The mirror fixing plate is made of colorless and transparent material. The mirror fixing plate 63 transmits the white laser beam emitted from the light-source unit 30.

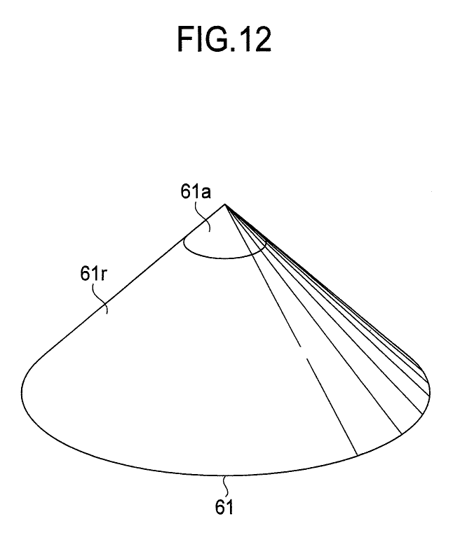

As shown in FIG. 12, the first mirror 61 is shaped as a cone. On the outside surface of the first mirror 61 is a first reflection surface 61r that reflects the white laser beam emitted from the light-source unit 30. In addition, near the apex of the cone on the outside surface of the first mirror 61 is also an attenuation surface 61a that attenuates the white laser beam.

The first mirror 61 is supported by the mirror fixing plate 63 via the connecting member 64 so that a conical axis of the first mirror 61 is perpendicular to the surface of the mirror fixing plate 63.

The optical unit 60 is attached to the hollow tube 57 so that the end having the smaller internal diameter faces the emission end of the illumination fiber 53 (see FIG. 5). In addition, the optical unit 60 is positioned so that the conical axis of the first mirror 61 is aligned with a first straight line (see “L1” in FIG. 5) that passes the standard point and is parallel to the axis direction of the hollow tube 57.

The white laser beam emitted from the illumination fiber 53 is reflected by the first reflection surface 61r of the first mirror 61 and reaches the second reflection surface of the second mirror 62. The white laser beam reaching the second reflection surface is reflected toward the mirror fixing plate 63 by the second reflection surface. The white laser beam reflected by the second reflection surface passes through the mirror fixing plate 63 and is shined on the observation area.

As described above, it is difficult to circulate or spirally move the emission end of the illumination fiber 53 in a stable manner within a circular area having a certain radius and the standard point as its center. A minimum radius that enables the emission end of the illumination fiber 53 to circulate in a stable manner is measured and defined as a first radius (first length).

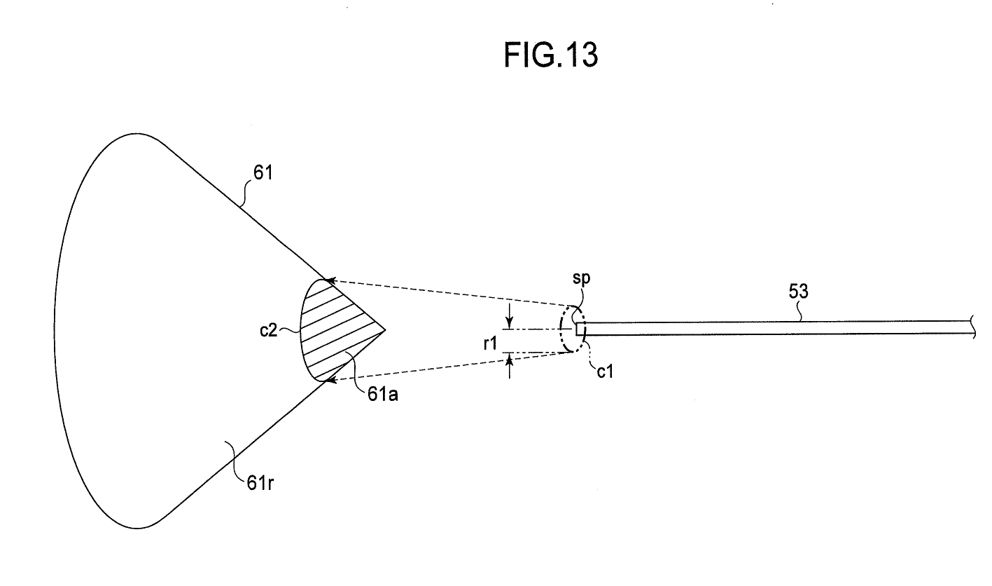

As shown in FIG. 13, the white laser beam emitted from the emission end that is moved along a first circumference (see “c1”) of a circular pattern with the standard point (see “sp”) at its center and the first radius (see “r1”) as its radius reaches a second circumference (see “c2”) on the first mirror 61. The second circumference is a locus defined by moving a point on the conical surface of the first mirror 61 so that the distance from the moved point to the apex remains constant.

The attenuation surface 61a (see shaded area) is formed on the conical surface bounded by the apex and the second circumference. In addition, the first reflection surface 61r is formed on the partial conical surface bounded by the second circumference and a circumference at the base of the conical first mirror 61.

In addition, the first and second mirrors 61 and 62 are formed so that the following formulas (1) and (2) are satisfied:

f1(θ1, θ2, θ3)=2×θ1−θ2−θ3<π/2 (1)

f2(θ1, θ2, θ3)=2×(θ1−θ2)−θ3>0 (2)

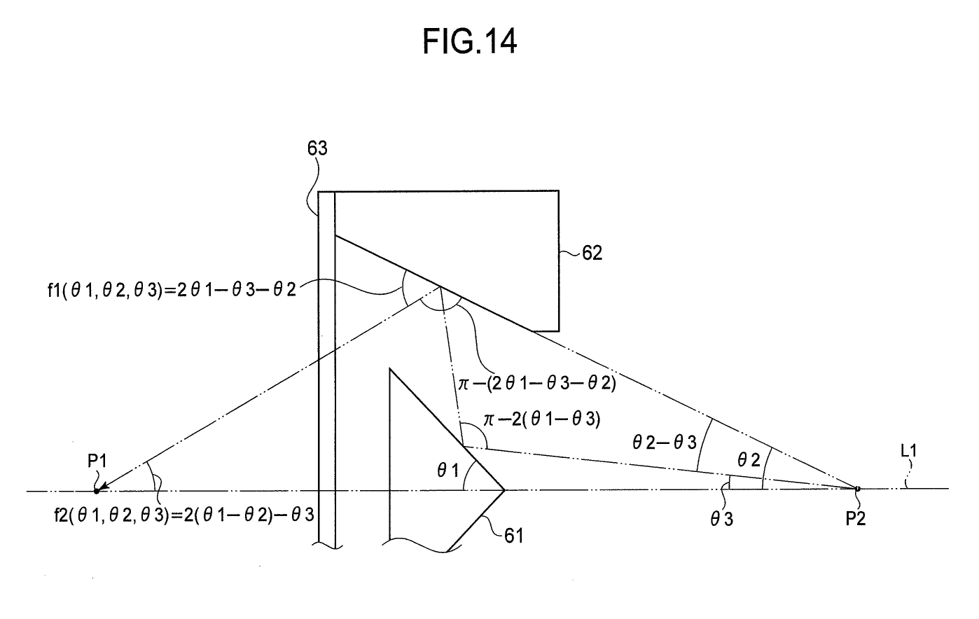

θ1 is an angle (first angle) between the first line (see “L1” in FIG. 14) and the generatrix line of the conical first mirror 61 in the above formulas. θ2 is an angle (second angle) between the first line and the generatrix line of the conical surface of the inside of the second mirror 62 in the above formulas. θ3 is an angle (third angle) between the first line and the emission direction of the white laser beam emitted from the emission end that is moved along the first circumference in the above formulas.

As shown in FIG. 14, f1 (θ1, θ2, θ3) is an angle between the forward direction of the white laser beam reflected by the second mirror 62 and the generatrix line of the conical surface on the inside of the second mirror 62 when the white laser beam is emitted from the emission end that is moved along the first circumference. Accordingly, by satisfying the formula (1), the white laser beam reaching the second mirror 62 from the first mirror 61 can be reflected towards the mirror fixing plate 63 (i.e., the direction including the first direction as a vector of a positive direction.

In addition, f2 (θ1, θ2, θ3) is an angle between the forward direction of the white laser beam reflected by the second mirror 62 and the first line when the white laser beam is emitted from the emission end that is moved along the first circumference. Accordingly, by satisfying the formula (2), the white laser beam reaching the second mirror 62 from the first mirror 61 can be reflected towards a first point (see “P1” in FIG. 14) on the first line when the white laser beam is emitted from the emission end that is moved along the first circumference. Consequently, the white laser beam can be shined on the entire area that is behind the first mirror 61 and on the other side from the illumination fiber 53.

However, the entire observation area is only observable if the observation area is a certain distance from a second point p2. The second point p2 is the point of intersection between the first line and the emission direction of the white laser beam from the emission end that is moved along the first circumference c1. The certain distance is the distance between the first point p1 and the second point p2.

The hood 56 is shaped as a cylindrical tube and holds the distal end of the insertion tube 51. The length of the hood 56 is determined so that the location of the observation area is in accordance with the first point p1. Using the hood 56 having the length determined according to the manner described here, an image can be reproduced with good quality by scanning the observation area with the white laser beam as the hood 56 is pressed to the observation area.

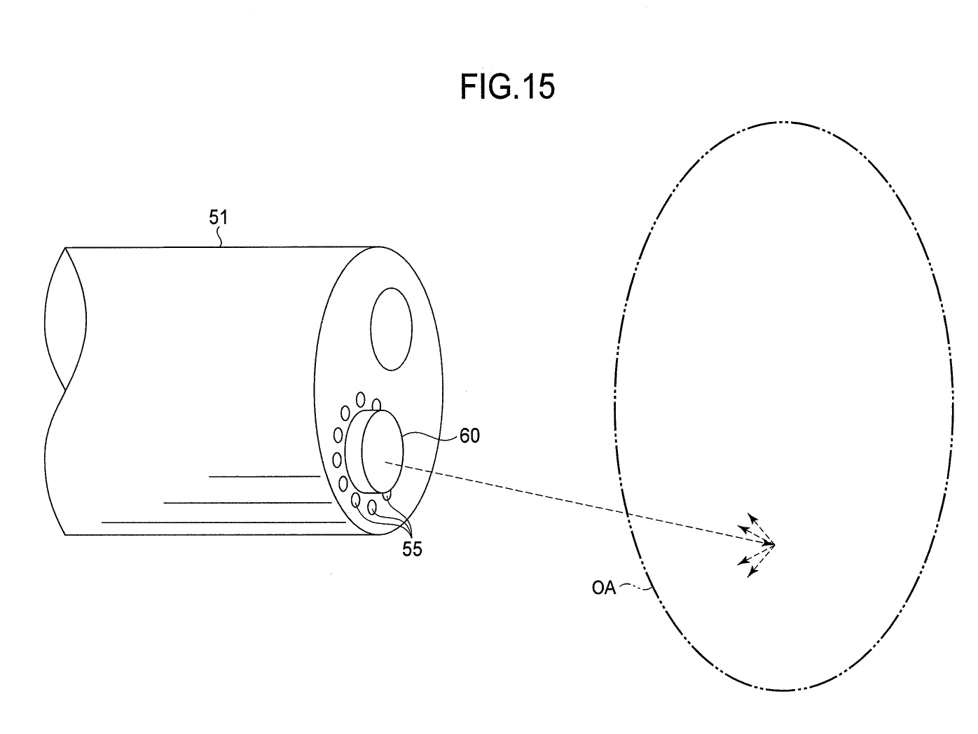

When the white laser beam is emitted from the illumination fiber 53 toward an individual point (see FIG. 15) within the observation area, the reflected light is scattered at the point. The scattered and reflected light is incident on the head end of the image fibers 55.

A plurality of the image fibers 55 are mounted in the scanning endoscope 50. The incident ends of the image fibers 55 are arranged around the optical unit 60. The light that is scattered and reflected from the point in the observation area is incident on all the image fibers 55.

The reflected light incident on the incident ends of the image fibers 55 is transmitted to the emission ends of the image fibers 55. As described above, the emission ends of the image fibers 55 are optically connected to the light-capturing unit 21. The reflected light transmitted to the emission ends is incident on the light-capturing unit 21.

The light-capturing unit 21 detects the amounts of red, green, and blue light components in the reflected light, and generates pixel signals according to the amounts of the light components. The pixel signals are transmitted to the image processing circuit 23.

The image processing circuit 23 estimates the points where the white laser beam is shined on the basis of signals used to control the scanner driver 22. In addition, the image processing circuit 23 stores the received pixel signals at the address of the image memory 26 that corresponds to the estimated points.

As described above, the observation area is scanned with the white laser beam, pixel signals are generated on the basis of the reflected light at the respective points illuminated with the white laser beam, and the generated pixel signals are stored at the addresses corresponding to the points. The image signal corresponding to the observation area comprises the pixel signals corresponding to the points from the scan-start point to the scan-end point. As described above, the image processing circuit 23 carries out predetermined image processing on the image signal. After undergoing predetermined image processing, the image signal is transmitted to the monitor 11.

In addition to the points where the white laser beam has been shined, the position of the emission end of the illumination fiber 53 is also estimated by the image processing circuit 23 on the basis of signals used to control the scanner driver 22. While the emission end of the illumination fiber 53 is moved along the first circumference, the emission of the white laser beam from the light-source unit 30, the generation of the pixel signals at the light-capturing unit 21, and the production of an image at the image processing circuit 23 are suspended.

In the above first embodiment, an image of the entire observation area can be produced without using the white laser beam emitted from the emission end that is moved near the center of the spiral course. By avoiding using the white laser beam emitted from the emission end that is moved near the center of the spiral course, distortion in the produced image will be reduced.

Next, a scanning endoscope and a scanning endoscope processor of the second embodiment are explained. The primary difference between the second embodiment and the first embodiment is the structure of the second mirror. In addition, in the second embodiment the position of the emission end of the illumination fiber is estimated on the basis of optical information gained from the scanning endoscope, unlike in the first embodiment. The second embodiment is explained mainly with reference to the structures that differ from those of the first embodiment. Here, the same index numbers are used for the structures that correspond to those of the first embodiment.

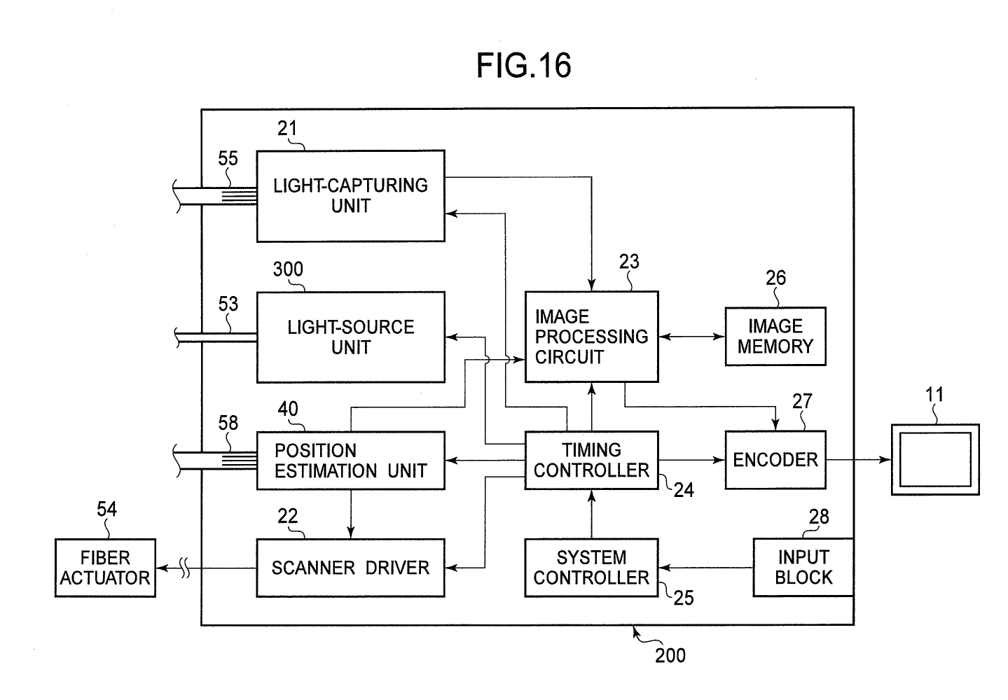

As shown in FIG. 16, the scanning endoscope processor 200 comprises a light-source unit 300, a light-capturing unit 21, a scanner driver 22, an image processing circuit 23, a timing controller 24, a system controller 25, and other components, as in the first embodiment. Further, the scanning endoscope processor 200 comprises a position estimation unit 40, unlike the first embodiment.

The light-source unit 300 provides the illumination fiber 53 with white light to illuminate an observation area, as in the first embodiment. In addition, the light-source unit 300 provides the illumination fiber 53 with ultraviolet light that is used for estimating the position of the emission end of the illumination fiber 53.

As described later, the ultraviolet light is emitted from the emission end of the illumination fiber 53, and transmitted to the position estimation unit 40 by a position detection fiber 58. The position estimation unit 40 estimates the position of the emission end of the illumination fiber 53. A position signal corresponding to the estimated position is then transmitted from the position estimation unit 40 to the scanner driver 22.

The scanner driver 22 controls the fiber actuator 54 to move the illumination fiber 53 on the basis of the position signal and a control signal transmitted from the position estimation unit 40 and the timing controller 24, respectively.

The reflected light at the point illuminated by the white laser beam emitted from the illumination fiber 53 is transmitted to the scanning endoscope processor 200 by the scanning endoscope (not depicted in FIG. 16), as in the first embodiment The transmitted light is made incident on the light-capturing unit 21.

The light-capturing unit 21 generates the image signal and stores it in the image memory 26, as in the first embodiment. The stored image signal is transmitted to the monitor 11 via the encoder 27, as in the first embodiment.

By connecting the scanning endoscope to the scanning endoscope processor 200, two optical connections are made: one between the light-source unit 300 and the illumination fiber 53, and the other between the light-capturing unit 21 and the image fibers 55, as in the first embodiment. In addition, by connecting the scanning endoscope to the scanning endoscope processor 200, the fiber actuator 54 is electrically connected with the scanning driver 22, as in the first embodiment. In addition, by connecting the scanning endoscope to the scanning endoscope processor 200, the position estimation unit 40 is optically connected with the position detection fibers 58 arranged in the scanning endoscope.

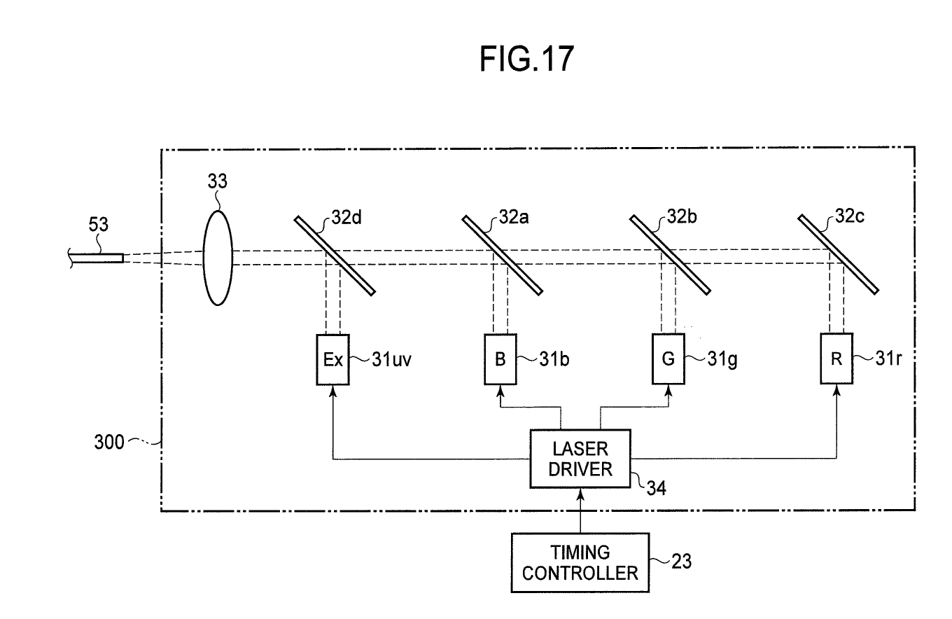

As shown in FIG. 17, the light-source unit 300 comprises a red laser 31r, a green laser 31g, a blue laser 31b, first to third filters 32a, 32b, and 32c, a condenser lens 33 and a laser driver 34, as in the first embodiment. In addition, the light-source unit 300 also comprises an ultraviolet laser 31uv and a fourth filter 32d, unlike the first embodiment.

The structures and functions of the red laser 31r, the green laser 31g, the blue laser 31b, the first to third filters 32a to 32c, the condenser lens 33, and the laser driver 34 are the same as those in the first embodiment.

The ultraviolet laser 31uv emits an ultraviolet laser beam with a wavelength that falls within the range of a first band. The first band is broad and different from the band of visible light. The fourth filter 32d reflects the ultraviolet light of the first band, and transmits the other bands. The fourth filter 32d is arranged between the condenser lens 33 and the first filter 32a.

The fourth filter 32d is fixed so that the surface of the filter is inclined by 45 degrees against the axis direction of the illumination fiber 53. The ultraviolet laser 31uv is mounted so that the ultraviolet laser beam emitted by the ultraviolet laser 31uv is reflected by the fourth filter 32d and made incident on the incident end of the illumination fiber 53. In addition, the ultraviolet laser is condensed by the condenser lens 33, and made incident on the incident end of the illumination fiber 53.

Upon observing a real-time image in the peripheral area of the insertion tube 51, the red, green, and blue laser beams are mixed into a white laser beam, which is supplied to the illumination fiber 53, as in the first embodiment. In addition, the ultraviolet light of the first band is supplied to the illumination fiber 53.

The laser driver 34 drives the red, green, and blue lasers 31r, 31g, and 31b, as in the first embodiment. In addition, the laser driver 34 drives the ultraviolet laser 31uv.

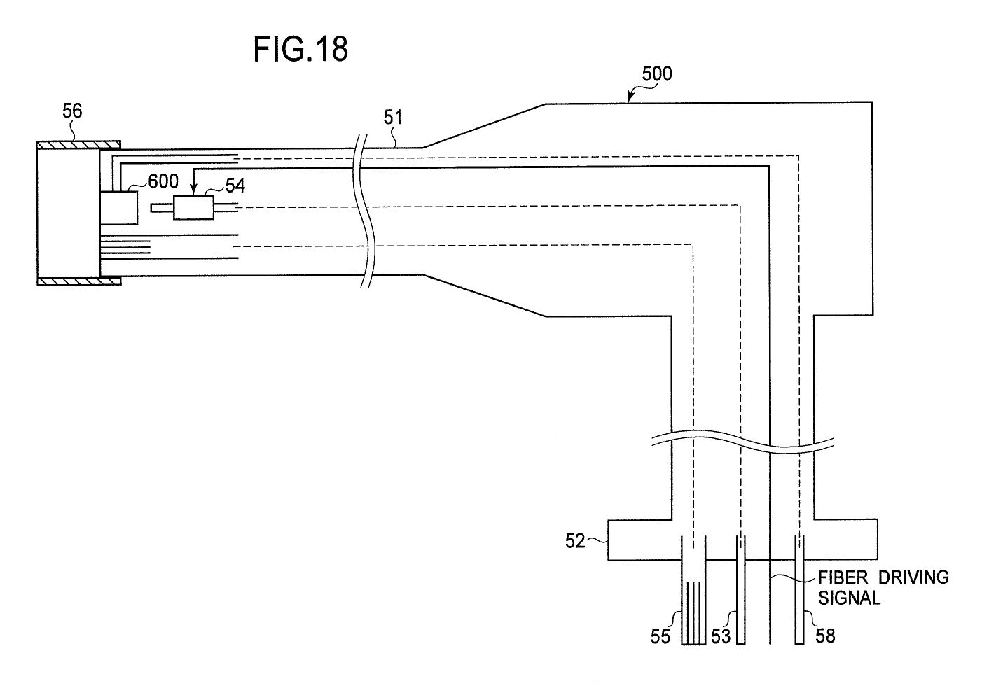

As shown in FIG. 18, the scanning endoscope 500 comprises the illumination fiber 53, the fiber actuator 54, the image fibers 55, an optical unit 600 and a hood 56, as in the first embodiment. In addition, the scanning endoscope 500 also comprises the position detection fiber 58.

The illumination fiber 53 and the image fibers 55 are arranged inside the scanning endoscope 500 from the connector 52 to the distal end of the insertion tube 51, as in the first embodiment. In addition, the position detection fiber 58 is arranged inside the scanning endoscope 500 from the connector 52 to the distal end of the insertion tube 51.

As described above, the white laser beam and the ultraviolet laser beam emitted by the light-source unit 300 is incident on the incident end of the illumination fiber 53. The incident white and ultraviolet laser beams are transmitted to the emission end of the illumination fiber 53.

A solid hollow tube 57 is mounted at the distal end of the insertion tube 51, as in the first embodiment (see FIG. 5). The illumination fiber 53 is supported inside of the hollow tube 57 by the fiber actuator 54. The posture of the hollow tube 57 for the insertion tube 51 and the posture of the illumination fiber for the hollow tube 57 are the same as those in the first embodiment.

The structure and the function of the fiber actuator 54 are the same as those in the first embodiment. The emission end of the illumination fiber 53 is moved along the spiral course on the basis of a fiber driving signal transmitted from the scanner driver 22, as in the first embodiment.

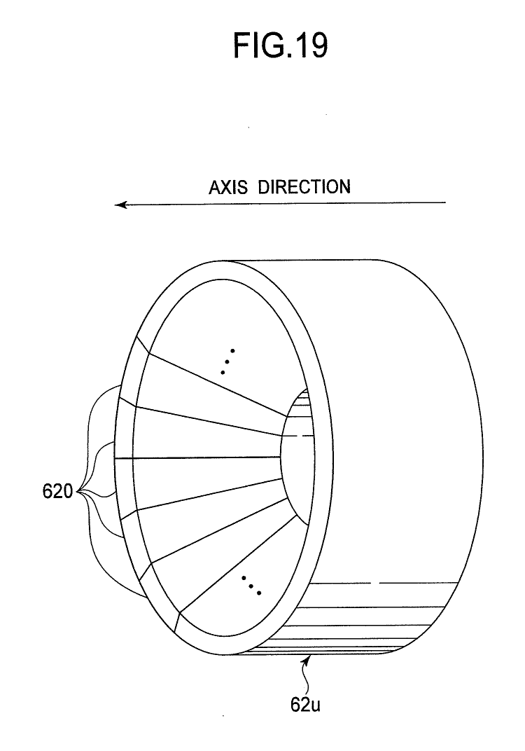

The optical unit 600 is mounted in the emission direction of light from the emission end that is positioned on the standard point, as in the first embodiment. The optical unit 600 comprises a first mirror 61, a second mirror 620 (see FIG. 19), and a mirror fixing plate 63, as in the first embodiment.

As shown in FIG. 19, a plurality of the second mirrors 620 forms a second mirror unit 62u, unlike the first embodiment. The shape of the second mirror unit 62u is the same as that of the second mirror 62 in the first embodiment. Accordingly, the second mirror unit 62u has a hollow tube shape so that the inside surface of the hollow tube is a conical surface, and the internal diameter of the hollow tube increases along the axis direction of the hollow tube shape. The inside conical surface of the second mirror unit 62u has a second reflection surface that reflects the white laser beam, which is visible light, and transmits the ultraviolet light of the first band.

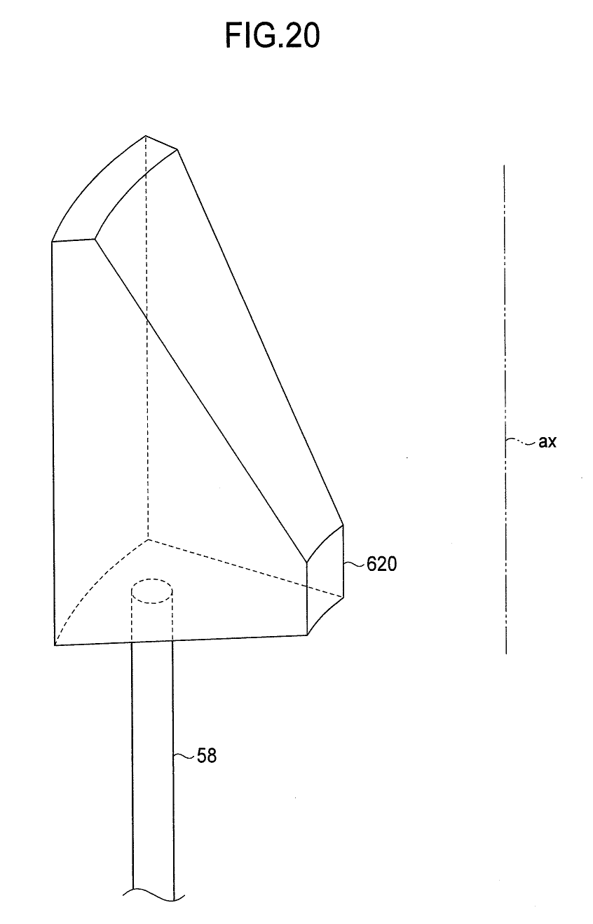

The second mirror 620 is shaped by dividing the second mirror unit 62u equally by planes that contain the axis (see “ax” in FIG. 20) of the hollow-tube-shaped second mirror unit 62u.

The second mirror 620 is connected to the position detection fiber 58. The ultraviolet light that reaches the inside of the second mirror 620 is incident on the position detection fiber 58 and is transmitted to the position estimation unit 40.

The surfaces of the second mirror 620 other than the second reflection surface reflect light of all bands. Accordingly, the ultraviolet light that reaches the second mirror 620 is repeatedly reflected by the surfaces except for the second reflection surface, and is incident on the position detection fiber 58. In addition, a plurality of position detection fibers 58 are individually connected, respectively, to each one of the plurality of second mirrors 620.

The mirror fixing plate 63 is attached to the second mirror unit 62u at the end having the greater internal diameter, as in the first embodiment. The structures, functions and arrangements of the mirror fixing plate 63 and the first mirror 61 are the same as those in the first embodiment. The optical unit 600 is attached to the hollow tube 57 so that the end having the smaller internal diameter faces the emission end of the illumination fiber 53, as in the first embodiment.

An angle (second angle) between the first line and the generatrix line of the conical surface inside the second mirror unit 62u is defined as θ2. The definitions of θ1 and θ3 are the same as those in the first embodiment. With such a definition, the first mirror 61 and the second mirror unit 62u are arranged so that the above formulas (1) and (2) are relevant, as in the first embodiment.

The structures and the functions of the hood 56 and the image fibers 55 are the same as those in the first embodiment. Accordingly, the reflected light of a fine point illuminated by the white laser beam is incident on the incident ends of the image fibers 55, and transmitted to the emission ends of the image fibers 55.

As described above, the ultraviolet light that reaches the second mirror 620 is transmitted to the position estimation unit 40 by the position detection fiber 58. In addition, the reflected light of the white laser beam illuminating the observation area is transmitted to the light-capturing unit 21 by the image fibers 54.

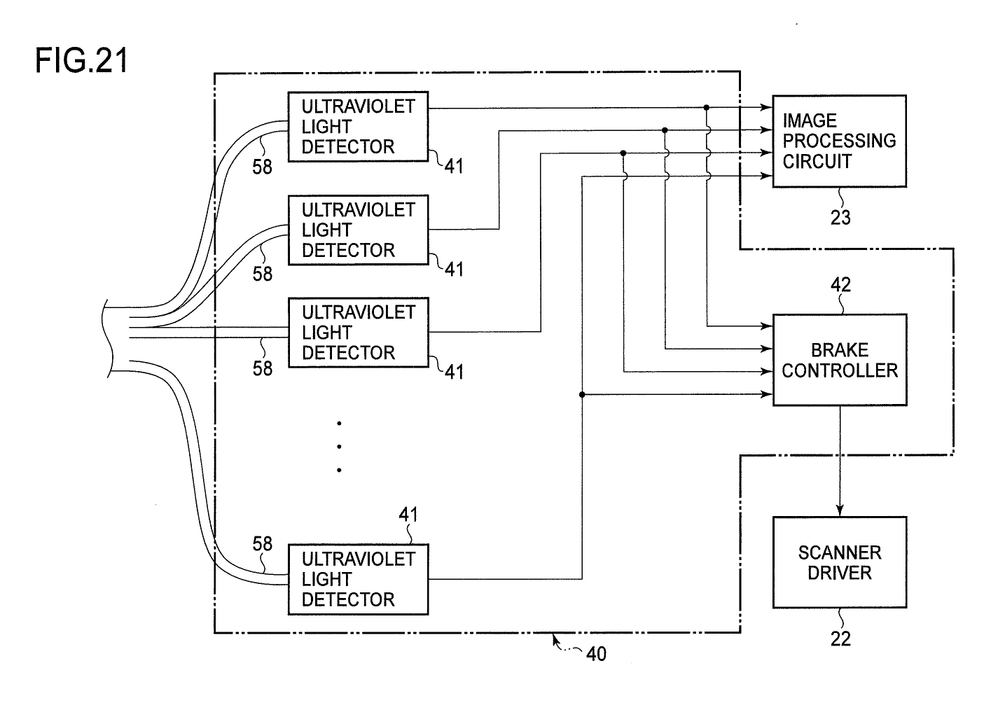

As shown in FIG. 21, the position estimation unit 40 comprises a plurality of ultraviolet light detectors 41 and a brake controller 42. Each of the ultraviolet light detectors 41 is optically connected to each of the position detection fibers 58. When detecting the capture of the ultraviolet light, the ultraviolet light detector 41 transmits a detection signal to the image processing circuit 23 and the brake controller 42.

The ultraviolet light emitted from the illumination fiber 53 is made incident on one of the plurality of second mirrors 620 that constitutes the second mirror unit 62u. Accordingly, the ultraviolet light is detected by only one of the many ultraviolet light detectors 41. Consequently, the inclined direction of the illumination fiber 53 is determinable on the basis of the ultraviolet light detector 41 that outputs the detection signal.

The brake controller 42 generates a braking signal in the braking period on the basis of the ultraviolet light detector 41 that outputs the detection signal, and transmits the braking signal to the scanner driver 22. The braking signal helps the emission end of the illumination fiber 53 to return along the first circumference. The scanner driver 22 generates the fiber driving signal on the basis of the braking signal, and transmits the fiber driving signal to the first and second bending elements 54b1 and 54b2.

The light-capturing unit 21 generates the pixel signals according to the amounts of reflected light, as in the first embodiment. The pixel signals are transmitted to the image processing circuit 23. The image processing circuit 23 estimates the points where the white laser beam is shined on the basis of the detection signal and the signals used to control the scanner driver 22. The image processing circuit 23 stores the received pixel signals at the address of the image memory 26 corresponding to the estimated points, as in the first embodiment.

The observation area is scanned with the white laser beam, pixel signals are generated on the basis of the light reflected from the respective points illuminated with the white laser beam, and the generated pixel signals are stored at the address corresponding to the points, as in the first embodiment. The image signal corresponding to the observation area comprises the pixel signals corresponding to the points from the scan-start point to the scan-end point. The image signal is transmitted to the monitor 11 after the image processing circuit 23 carries out predetermined image processing on the image signal, as in the first embodiment.

While the emission end of the illumination fiber 53 is moved within the first circumference, the emission of the white laser beam from the light-source unit 30, the generation of the pixel signals at the light-capturing unit 21, and the production of an image at the image processing circuit 23 are all suspended, as in the first embodiment.

In the above second embodiment, an image of the entire observation area can be produced without using the white laser beam emitted from the emission end that is moved near the center of the spiral course. By avoiding using the white laser beam, distortion in the produced image will be reduced.

In addition, in the above second embodiment, it is possible to detect the direction in which the emission end of the illumination fiber 53 inclines. Accordingly, the time required to return the emission end of the illumination fiber 53 to the first circumference, from the start of the braking period, can be shortened.

In addition, the accuracy of the estimation of the points where the white laser beam is shined is improved because the point is estimated using the direction in which the emission end of the illumination fiber 53 inclines. It is possible to reduce the influence of the distortion appearing in the displayed image by improving the accuracy of the estimation.

The first mirror 61 is shaped as a cone in the first and second embodiments. However, the shape of the first mirror 61 is not limited to a cone. Other shapes can be adopted as long as the distance from the first position on the first straight line to any second position on the first reflection surface increases with the distance between the first position and the illumination fiber 53. The line connecting the first and second positions is perpendicular to the first straight line. In other words, other shape can be adopted as long as the distance from the first position to any second position increases as the first position is moved toward the first direction. For example, bowl and bell shapes can be adopted.

The inside of the hollow tube of the second mirror 62 and 620 is shaped conically, in the first and second embodiments. However, the inside shape of the second mirrors is not limited to a conical shape. The same effect as the above embodiments can be achieved as long as the second mirror is shaped so that the light reflected by the first mirror 61 is in the direction that includes the first direction as a positive vector, and is toward any point on the first line.

The emission end of the illumination fiber 53 is moved by inclining the illumination fiber 53, in the first and second embodiments. However, the emission end can be moved without inclining the illumination fiber 53. If the emission end is moved without inclining the illumination fiber 53, the direction in which the white laser beam is emitted from the emission end is parallel to the first direction. In such a case, the same effect as the above embodiments can be achieved by shaping the first mirror 61 and the second mirror 62 and 620 so that the first angle is greater than the second angle.

The second mirror 62 and 620 entirely surrounds the first reflection surface of the first mirror 61, in the first and second embodiments. However, the first reflection surface may not be surrounded entirely. Even if the first reflection surface is not surrounded, the observation area can be scanned with the white laser beam.

The distal end of the insertion tube 51 is held by the hood 56, in the first and second embodiments. However, the observation area can be observed without the hood 56. Without the hood 56, it is possible for a user to produce an accurate image by adjusting the distance from the distal end of the insertion tube 51 to the observation area.

The first mirror 61 has the attenuation surface 61a, in the first and second embodiments. However, the first mirror 61 may not have the attenuation surface 61a. Unless the first mirror 61 has the attenuation surface 61a, the observation area is scanned with the white laser beam emitted from the emission end, which moves along the first circumference in an unstable manner. However, it is still possible to produce an accurate image because the production of the image is suspended while the emission end is moved along the first circumference. Because of the attenuation surface 61a in the above embodiments, the white laser beam, which is unnecessary for illumination, is prevented from being shined on the observation area.

The emission of the white laser beam from the light-source unit 30 and 300 is suspended when the emission end of the illumination fiber 53 is moved along the first circumference, in the first and second embodiments. However, the emission may not be suspended. As described above, it is possible to produce an accurate image even if the emission is not suspended, because the production of the image is suspended while the emission end is moved along the first circumference. Owing to the suspension of the emission as in the above embodiments, the power consumption can be reduced.

The generation of the pixel signals by the light-capturing unit 21 is suspended when the emission end of the illumination fiber 53 is moved along the first circumference, in the first and second embodiments. However, the generation may not be suspended. As described above, even if the pixel signals are generated when the emission end is moved along the first circumference, the pixel signals are not used for the production of the image signal because the production of the image is suspended while the emission end is moved along the first circumference. Accordingly, it is possible to produce an accurate image even if the generation is not suspended. Owing to the suspension of the generation of the pixel signals as in the above embodiments, the power consumption can be reduced.

The first band pertains to a range of wavelengths for ultraviolet light, in the second embodiment. However, the first band may be a range of wavelengths of infrared light. Or, the first band can be a range for any light with a wavelength that is different from those of the red, green and blue lights emitted by the red, green, and blue lasers 31r, 31g, and 31b, respectively, and which passes through the second mirror 620.

The detection signal output by the ultraviolet light detector 41 is used for the estimation of the points where the white laser beam is shined, in the second embodiment. However, the detection signal may not be used for the estimation.

Lasers are used as light sources to emit red, green, and blue light, in the first and second embodiments. However, other kinds of light sources may be used. But, a laser is preferable for the light source in the above embodiments because it is preferable to shine the illumination light on a minute point within an observation area of the scanning endoscope, and a laser can emit light having strong directivity.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2008-289209 (filed on Nov. 11, 2008), which is expressly incorporated herein, by reference, in its entirety.

1. A scanning endoscope comprising:
a first transmitter that emits a beam of radiant light from an emission end, the beam of radiant light being shined on an observation area,
an actuator that moves the emission end along a spiral course from a predetermined standard point;
a first mirror that is arranged from the emission end toward a first direction when the emission end is on the standard point, the radiant light being emitted in the first direction from the emission end when the emission end is on the standard point, the first mirror comprising a first reflection surface around the first direction, the distance between a first position on a first line and any second position on the first reflection surface increasing as the first position is moved in the first direction, the standard point being on the first line, the first line being parallel to the first direction, the first reflection surface reflecting the radiant light emitted from the first transmitter, a line connecting the first and second positions being perpendicular to the first line; and
a second mirror that is arranged around the first reflection surface, the second mirror comprising a second reflection surface, the second reflection surface reflecting the radiant light reflected by the first mirror in a direction that includes the first direction as a positive vector toward any points on the first line.

2. A scanning endoscope according to claim 1, wherein the first and second mirrors are formed so that a first angle is greater than a second angle, the first angle is an angle between the first line and a tangent line of any points on the first reflection surface, and the second angle is an angle between the first line and a tangent line of a point of the second reflection surface on which the radiant light is reflected from the first reflection surface.

3. A scanning endoscope according to claim 2, wherein,
the actuator moves the emission end by bending a part of the first transmitter, and
the first and second mirrors are shaped so that the formulas of 2×θ1−θ2−θ3<π/2 and 2×(θ1−θ2)−θ3>0 are satisfied, θ1 being the first angle at a point that the radiant light reaches when the first transmitter is inclined by a predetermined third angle of θ3 from the first line, θ2 being the second angle at a point that the radiant light reflected by the first reflection surface reaches when the first transmitter is inclined by the third angle from the first line.

4. A scanning endoscope according to claim 1, further comprising a second transmitter that transmits a component of the radiant light passing through the second reflection surface, the second transmitter being optically connected to the second mirror, a plurality of the second mirrors being arranged around the first reflection surface.

5. A scanning endoscope according to claim 4, wherein a plurality of the second mirrors are arranged around the entire first reflection surface.

6. A scanning endoscope according to claim 1, wherein the second mirror surrounds the first reflection surface.

7. A scanning endoscope according to claim 1, further comprising a guide that maintains a consistent distance between the emission end and the observation area so that a point of the observation area illuminated with the radiant light reflected by the first and second mirrors is on the first line when the emission end is moved from the standard point by a first length.

8. A scanning endoscope according to claim 7, wherein an attenuation surface is formed on the first mirror, the attenuation surface attenuating the radiant light emitted from the emission end when the emission end is within a first area, the center of the first area being the standard point, the radius of the first area being the first length.

9. A scanning endoscope according to claim 1, wherein the first reflection surface is parallel to a side surface of a circular truncated cone of which its axis is the first line.

10. A scanning endoscope according to claim 1, wherein the second reflection surface is parallel to a side surface of a circular truncated cone of which its axis is the first line.

11. A scanning endoscope processor comprising:
a light source that supplies the radiant light to the first transmitter of the scanning endoscope of claim 1;
a light receiver that detects and receives various amounts of reflected light at the observation area illuminated with the radiant light;
an image processor that produces an image corresponding to the observation area on the basis of the amounts of the reflected light detected by the light receiver; and
a first controller that suspends the production of an image at the image processor when the emission end is within a first area of which its center is the standard point and of which its radius is a first length, the first controller ordering the image processor to produce the image when the emission end is outside of the first area.

12. A scanning endoscope processor according to claim 11, further comprising a second controller that suspends the emission of the radiant light from the light source when the emission end is within the first area, the second controller ordering the light source to emit the radiant light when the emission end is outside of the first area.

13. A scanning endoscope processor according to claim 11, further comprising a third controller that suspends the detection of the various amounts of the reflected light by the light receiver when the emission end is within the first area, the third controller ordering the light receiver to detect the various amounts of the reflected light when the emission end is outside of the first area.

14. A scanning endoscope processor according to claim 11, further comprising:
a light detector that detects light of a first band from one of a plurality of second transmitters, the scanning endoscope comprising a plurality of the second transmitters, the second transmitters being connected to a plurality of the second mirrors, a plurality of the second mirrors being arranged around the first reflection surface, the light of the first band incident on the second reflection surface passing through the second reflection surface, light of a second band, which is light of the other band, being reflected by the second surface, the second transmitters transmitting the light of the first band passing through the second reflection surface; and
a location estimator that estimates the location of the emission end on the basis of the second transmitter from which the light detector detects the light of the first band,
the light source supplying the light of the first band in addition to the radiant light to the first transmitter.

15. A scanning endoscope apparatus comprising a scanning endoscope according to claim 1 and a scanning endoscope processor comprising:
a light source that supplies the radiant light to the first transmitter of the scanning endoscope of claim 1;
a light receiver that detects and receives various amounts of reflected light at the observation area illuminated with the radiant light;
an image processor that produces an image corresponding to the observation area on the basis of the amounts of the reflected light detected by the light receiver; and
a first controller that suspends the production of an image at the image processor when the emission end is within a first area of which its center is the standard point and of which its radius is a first length, the first controller ordering the image processor to produce the image when the emission end is outside of the first area.
